(19) United States
(12) Patent Application Publication
(10) Pub. No.: US 2025/0318454 A1
(43) Pub. Date: Oct. 16, 2025

(54) SYSTEMS AND METHODS FOR AN AGRICULTURAL SYSTEM

(71) Applicant: RAVEN INDUSTRIES, INC., Sioux Falls, SD (US)

(72) Inventor: Jared Ernest Kocer, Sioux Falls, SD (US)

(21) Appl. No.: 18/634,094

(22) Filed: Apr. 12, 2024

(51) Int. Cl. A01B 69/04; B60K 35/22; B60K 35/28; G01N 21/31; G01N 33/24

(52) U.S. Cl. CPC A01B 69/008; B60K 35/22; B60K 35/28; G01N 21/31; G01N 33/245; B60K 2360/166

(57) ABSTRACT

An agricultural system includes a vehicle including one or more ground tractive elements. A field sensor may be configured to capture data indicative of a moisture content within a field. A computing system may be communicatively coupled to the field sensor. The computing system may be configured to receive data from the field sensor, identify one or more zones of the field having a moisture content that exceeds a defined moisture content, calculate a probability of the vehicle experiencing tractive element slippage while traversing through the one or more zones, and generate a control command based at least in part on the probability of the vehicle experiencing tractive element slippage within the one or more zones.

### FIELD OF THE INVENTION

The present subject matter relates generally to agricultural vehicles that may be operated within an agricultural field.

### BACKGROUND OF THE INVENTION

Agricultural vehicles may traverse a field to perform various operations, such as a tillage operation, a seeding operation, an application operation, a harvesting operation, and/or any other operation. In some cases, various field conditions may affect the efficiency and/or outcome of the operation. For example, during various operations, an amount of moisture content within a field may cause issues for the vehicle traversing the field. Accordingly, an improved system and method for detecting one or more field conditions would be welcomed in the technology.

### BRIEF DESCRIPTION OF THE INVENTION

Aspects and advantages of the technology will be set forth in part in the following description, or may be obvious from the description, or may be learned through practice of the technology.

In some aspects, the present subject matter is directed to an agricultural system that includes a vehicle including one or more ground tractive elements. A field sensor is configured to capture data indicative of a moisture content within a field. A computing system communicatively coupled to the field sensor. The computing system includes a processor and associated memory. The memory stores instructions that, when implemented by the processor, configure the computing system to receive the data from the field sensor; identify one or more zones of the field having a moisture content that exceeds a defined moisture content; calculate a probability of the vehicle experiencing tractive element slippage while traversing through the one or more zones, and generate a control command based at least in part on the probability of the vehicle experiencing tractive element slippage exceeding a defined probability value within the one or more zones.

In some aspects, the present subject matter is directed to a method for operating an agricultural system. The method includes receiving data from a field sensor. The method also includes identifying, with a computing system, one or more zones of a field having a moisture content that exceeds a defined moisture content based on data from the field sensor. Lastly, the method includes calculating, with the computing system, a probability of a vehicle experiencing tractive element slippage exceeding a defined probability value while traversing through the one or more zones.

In some aspects, the present subject matter is directed to an agricultural system that includes a field sensor configured to capture data indicative of a moisture content within a field. A computing system is communicatively coupled to the field sensor. The computing system includes a processor and associated memory. The memory stores instructions that, when implemented by the processor, configure the computing system to receive data from the field sensor; identify one or more zones of the field having a moisture content that exceeds a defined moisture content, and calculate a probability of a vehicle experiencing tractive element slippage exceeding a defined probability value while traversing through the one or more zones.

These and other features, aspects, and advantages of the present technology will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present technology, including the best mode thereof, directed to one of ordinary skill in the art, is set forth in the specification, which makes reference to the appended figures, in which:

FIG. 1 illustrates a perspective view of an agricultural vehicle in accordance with aspects of the present subject matter;

FIG. 2 illustrates a side view of the vehicle in accordance with aspects of the present subject matter;

FIG. 3 is a schematic representation of a system including various vehicles in accordance with aspects of the present subject matter;

FIG. 4 illustrates a block diagram of components of a system for an agricultural vehicle in accordance with aspects of the present subject matter; and

FIG. 5 illustrates a flow diagram of a method for operating an agricultural system in accordance with aspects of the present subject matter.

Repeat use of reference characters in the present specification and drawings is intended to represent the same or analogous features or elements of the present technology.

### DETAILED DESCRIPTION OF THE INVENTION

Reference now will be made in detail to embodiments of the disclosure, one or more examples of which are illustrated in the drawings. Each example is provided by way of explanation of the discourse, not limitation of the disclosure. In fact, it will be apparent to those skilled in the art that various modifications and variations can be made in the present disclosure without departing from the scope or spirit of the disclosure. For instance, features illustrated or described as part can be used with another embodiment to yield a still further embodiment. Thus, it is intended that the present disclosure covers such modifications and variations as come within the scope of the appended claims and their equivalents.

In this document, relational terms, such as first and second, top and bottom, and the like, are used solely to distinguish one entity or action from another entity or action, without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

As used herein, the terms “first,” “second,” and “third” may be used interchangeably to distinguish one component from another and are not intended to signify a location or importance of the individual components. The terms “coupled,” “fixed,” “attached to,” and the like refer to both direct coupling, fixing, or attaching, as well as indirect coupling, fixing, or attaching through one or more intermediate components or features, unless otherwise specified herein. The terms “upstream” and “downstream” refer to the relative direction with respect to an agricultural product within a fluid circuit. For example, “upstream” refers to the direction from which an agricultural product flows, and “downstream” refers to the direction to which the agricultural product moves. The term “selectively” refers to a component's ability to operate in various states (e.g., an ON state and an OFF state) based on manual and/or automatic control of the component.

Furthermore, any arrangement of components to achieve the same functionality is effectively “associated” such that the functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected” or “operably coupled” to each other to achieve the desired functionality, and any two components capable of being so associated can also be viewed as being “operably couplable” to each other to achieve the desired functionality. Some examples of operably couplable include, but are not limited to, physically mateable, physically interacting components, wirelessly interactable, wirelessly interacting components, logically interacting, and/or logically interactable components.

The singular forms “a,” “an,” and “the” include plural references unless the context clearly dictates otherwise.

Approximating language, as used herein throughout the specification and claims, is applied to modify any quantitative representation that could permissibly vary without resulting in a change in the basic function to which it is related. Accordingly, a value modified by a term or terms, such as “about,” “approximately,” “generally,” and “substantially,” is not to be limited to the precise value specified. In at least some instances, the approximating language may correspond to the precision of an instrument for measuring the value, or the precision of the methods or apparatus for constructing or manufacturing the components and/or systems. For example, the approximating language may refer to being within a ten percent margin.

Moreover, the technology of the present application will be described in relation to exemplary embodiments. The word “exemplary” is used herein to mean “serving as an example, instance, or illustration.” Any embodiment described herein as “exemplary” is not necessarily to be construed as preferred or advantageous over other embodiments. Additionally, unless specifically identified otherwise, all embodiments described herein should be considered exemplary.

As used herein, the term “and/or,” when used in a list of two or more items, means that any one of the listed items can be employed by itself, or any combination of two or more of the listed items can be employed. For example, if a composition or assembly is described as containing components A, B, and/or C, the composition or assembly can contain A alone; B alone; C alone; A and B in combination; A and C in combination; B and C in combination; or A, B, and C in combination.

As used throughout this disclosure, the term “autonomous” refers to a vehicle capable of implementing at least one operation without driver input. An “operation” refers to a change in one or more of the steering, braking, acceleration/deceleration of the vehicle, actuation of a component of an implement, actuation of a component of a trailer, and/or actuation of any other component of the vehicle and/or any assembly operably coupled with the vehicle. The term “semi-autonomous” refers to a vehicle capable of implementing at least one operation that is not fully automatic but assists the operator with such operation (e.g., fully operational without a driver or driver input). As such an autonomous vehicle includes those that can operate under operator control during certain time periods and without operator control during other time periods while a semi-autonomous vehicle includes those that can operate under operator control during certain time periods and assist with operator control during other time periods.

As used herein, an unmanned aerial vehicle (UAV) may be any vehicle capable of being flown over a defined area. The UAV may be operated manually from a remote location, capable of autonomous operation, and/or capable of semi-autonomous operation at various times. Moreover, the UAV may be human-controlled, autonomously controlled, and/or semi-autonomously controlled without departing from the teachings provided herein.

In general, the present subject matter is directed to an agricultural system that includes a vehicle including one or more ground tractive elements. A field sensor may be configured to capture data indicative of a moisture content within a field. In some cases, the field sensor may be configured as a hyperspectral sensor. In such instances, the data collected from the hyperspectral sensor may be associated with a reflectivity value of the soil within the field.

A computing system may be communicatively coupled to the field sensor. The computing system may be configured to receive data from the field sensor, identify one or more zones of the field having a moisture content that exceeds a defined moisture content, calculate a probability of the vehicle experiencing tractive element slippage while traversing through the one or more zones, and generate a control command based at least in part on the probability of the vehicle experiencing tractive element slippage exceeding a defined probability value within the one or more zones. The system provided herein may reduce negative affects to the field and/or the probability of the vehicle getting stuck or generally failing to continue traversing the field at a defined speed during operation. Likewise, when the vehicle is operably coupled with an implement that includes a ground-contacting component, such as a ground engaging tool (e.g., tillage tools), the system provided herein may consider that ground-engaging tool that is used to also reduce the negative affects to the field and/or the probability of the vehicle getting stuck or generally failing to continue traversing the field at a defined speed during operation.

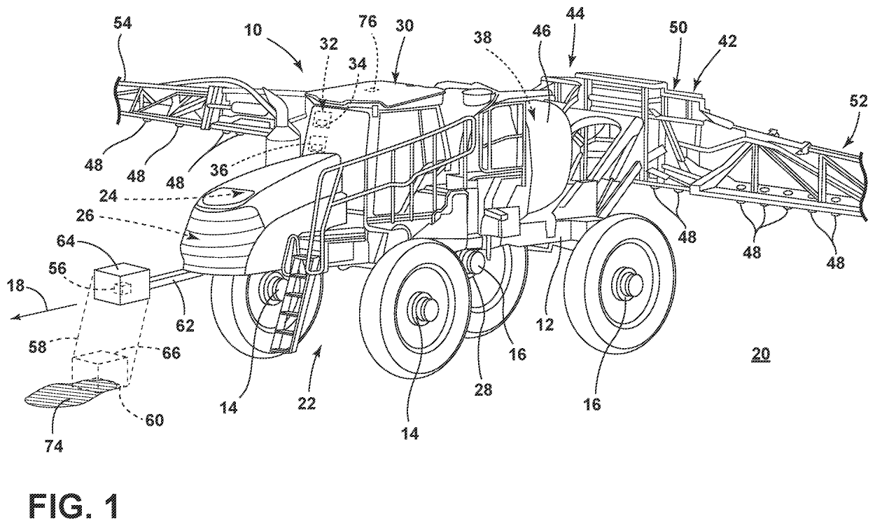

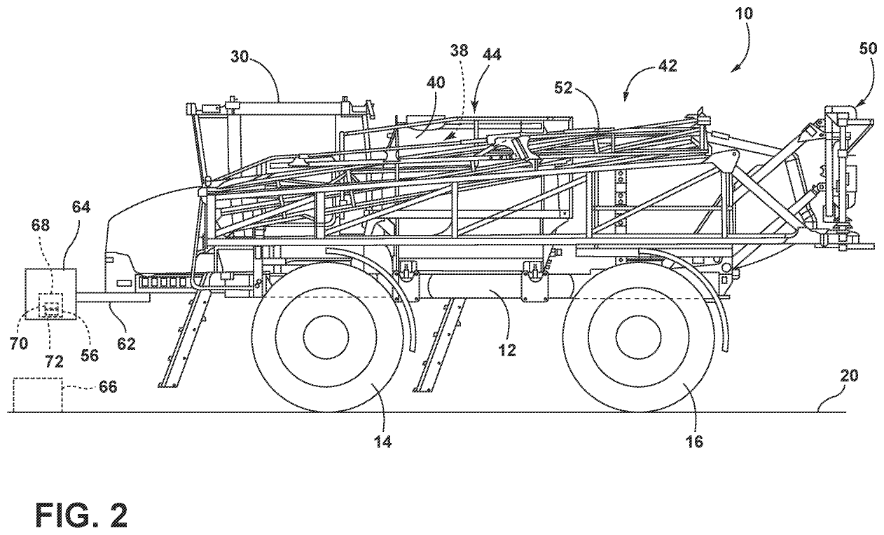

Referring now to FIGS. 1 and 2, a vehicle 10 is generally illustrated as a self-propelled agricultural applicator. However, in alternate embodiments, the vehicle 10 may be configured as any other suitable type of vehicle 10 configured to perform agricultural application operations, such as a tractor, a harvester, a self-propelled windrower, a self-propelled sprayer, and/or the like. In addition, the vehicle 10 may be operable coupled with an implement. For example, the implement may be configured as any suitable type of implement, such as a tillage implement or a planter. Furthermore, the vehicle 10 may correspond to any suitable powered and/or unpowered vehicle 10 (including suitable equipment, such as only a work vehicle or only an implement). Additionally, the vehicle 10 may include two or more associated pieces of equipment, implements, and/or the like (e.g., a tractor, a planter, and an associated air cart). In addition, it will be appreciated that the vehicle 10 may be human-controlled, autonomously controlled, and/or semi-autonomously controlled without departing the scope of the present disclosure.

In various embodiments, the vehicle 10 may include a chassis 12 configured to support or couple to a plurality of components. For example, tractive elements, such as front and rear wheels 14, 16 may be coupled to the chassis 12. The wheels 14, 16 may be configured to support the vehicle 10 relative to a field 20 and move the vehicle 10 in a direction of travel (e.g., as indicated by arrow 18 in FIG. 1) across the field 20. In this regard, the vehicle 10 may include a powertrain control system 22 that includes a power plant 24, such as an engine, a motor, or a hybrid engine-motor combination, a hydraulic propel or transmission system 26 configured to transmit power from the power plant 24 to the wheels 14, 16, and/or a brake system 28.

The chassis 12 may also support a cab 30, or any other form of user's station, permitting the user to control the operation of the vehicle 10. For instance, as shown in FIG. 1, the vehicle 10 may include a user interface 32 having a display 34 for providing messages and/or alerts to the user and/or for allowing the user to interface with the vehicle's controller through one or more user input devices 36 (e.g., levers, pedals, control panels, buttons, and/or the like).

The chassis 12 may also support a boom assembly 42 mounted to the chassis 12. In addition, the chassis 12 may support a product application system 44 that includes one or more tanks 46, such as a rinse tank and/or a product tank. The product tank is generally configured to store or hold an agricultural product 38, such as a pesticide, a fungicide, a rodenticide, a nutrient, and/or the like. The agricultural product 38 is conveyed from the product tank through plumbing components, such as interconnected pieces of tubing, for release onto the underlying field 20 (e.g., plants and/or soil) through one or more nozzle assemblies 48 mounted on the boom assembly 42.

As shown in FIGS. 1 and 2, the boom assembly 42 can include a frame 50 that supports first and second boom arms 52, 54, which may be orientated in a cantilevered nature. The first and second boom arms 52, 54 are generally movable between an operative or unfolded position (FIG. 1) and an inoperative or folded position (FIG. 2). When distributing the product, the first and/or second boom arm 52, 54 extends laterally outward from the vehicle 10 to cover swaths of the underlying field 20, as illustrated in FIG. 1. However, to facilitate transport, each boom arm 52, 54 of the boom assembly 42 may be independently folded forwardly or rearwardly into the inoperative position, thereby reducing the overall width of the vehicle 10, or in some examples, the overall width of a towable implement when the applicator is configured to be towed behind the vehicle 10.

Furthermore, in accordance with aspects of the present subject matter, the agricultural vehicle 10 may include one or more field sensor(s) 56 coupled thereto and/or supported thereon. Each field sensor(s) 56 may, for example, be configured to capture data relating to one or more field conditions of the field 20 along which the vehicle 10 is being traversed. For example, in several examples, the field sensor(s) 56 may be used to collect data associated with one or more features of the field 20, such as one or more conditions relating to moisture content, crop residue, soil clods, and/or surface irregularities (e.g., ridges and/or valleys) within the field 20. For instance, as will be described below, the field sensor(s) 56 may be used to collect data associated with a reflectivity value of the soil within the field 20. The measured reflectivity values may then be used as input into a predetermined model (e.g., a machine-learned model) for identifying one or more zones 74 of the field 20 having a moisture content that exceeds a defined moisture content.

With further reference to FIGS. 1 and 2, the field sensor(s) 56 may be provided in operative association with the agricultural vehicle 10 such that the field sensor(s) 56 has a field of view 58 directed towards a region(s) 60 of the field 20 adjacent to the vehicle 10, such as a region(s) 60 of the field 20 disposed in front of, behind, and/or along one or both of the sides of the vehicle 10. For example, as shown in FIG. 1, in some embodiments, a field sensor(s) 56 may be provided at a forward end portion of the vehicle 10 to allow the field sensor(s) 56 to capture images and related data of a section of the field 20 disposed in front of the work vehicle 10. Such a forward-located field sensor(s) 56 may capture data indicative of the field 20 before the vehicle 10 traverses such region(s) 60. Additionally or alternatively, the field sensor(s) 56 may be installed at any other suitable location(s) on the vehicle 10.

In some embodiments, a suitable mounting structure 62 (e.g., mounting arms, brackets, trays, etc.) may be used to support each field sensor(s) 56 forwardly of the cab 30 of the vehicle 10 (e.g., in a cantilevered arrangement) to allow the field sensor(s) 56 to obtain the desired field of view 58, including the desired orientation of the device's field of view 58 relative to the field 20. In some cases, a housing 64 supports the field sensor(s) 56 and may be operably coupled with the mounting structure 62.

Referring further to FIGS. 1 and 2, in general, the field sensor(s) 56 may correspond to any suitable device(s) or other assembly configured to capture data the associated with the field 20. For instance, the field sensor(s) 56 may be configured as a hyperspectral sensor that is configured to generate hyperspectral image data. Hyperspectral image data can be data that includes multiple spectral region(s) 60 to image the region(s) 60 of the field 20. Specifically, each particular region(s) 60 can have a unique spectral signature extending across multiple bands of the electromagnetic spectrum. This spectral signature contains field information about the corresponding region(s) 60 of the field 20.

In some examples, a “hyperspectral data cube” 66 is generated by the hyperspectral sensor that includes a spectrum corresponding to each region(s) 60. The spectra are stored within a three-dimensional volume, in which two of the axes represent the x- and y-coordinates of the region(s) 60, and the third axis represents the wavelengths within the corresponding spectra. The intensity at a particular point within the cube 66 can represent the intensity of a particular wavelength at a particular region(s) 60 having coordinates (x, y).

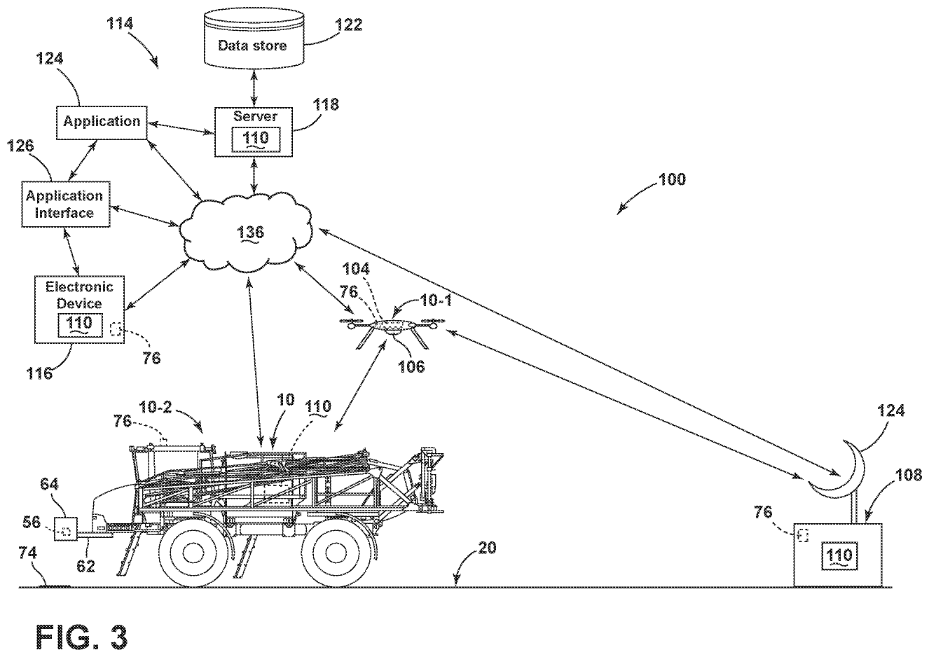

In some cases, the hyperspectral sensor can store each cube 66 in a sensor storage device 68, and then pass the cube 66 to a computing system 110 (FIG. 3). In other embodiments, the sensor can provide hyperspectral data planes to the computing system 110 (FIG. 3), which then constructs, stores, and processes the hyperspectral data cubes 66. The spectra corresponding to the region(s) 60 can be stored in any other suitable format, or at any other suitable location (e.g., stored remotely).

The hyperspectral sensor can include a charge-coupled device (CCD) 70 or other appropriate sensor that generates a digital signal representing the spectrum. The CCD 70 may be arranged at a fixed distance from a dispersive optic 72. The distance between the CCD 70 and the dispersive optic 72, together with the size of the sensor elements that make up the CCD 70, can determine (in part) the spectral resolution of the hyperspectral sensor. The spectral resolution, which is the width (e.g., full width at half maximum, or FWHM) of the component wavelengths collected by the sensor, may be selected to be sufficiently small to capture spectral features of field conditions of interest. The sensed intensity of component wavelengths depends on many factors, including the light source intensity, the sensor sensitivity at each particular component wavelength, the reflectance or transmittance of different sensor components such as scan mirror, polarizer, lens, and dispersive optic, and the exposure time of the sensor element to the component wavelength. These factors are selected such that the sensor is capable of sufficiently determining the intensity of component wavelengths so that it can distinguish the spectral features of field conditions of interest.

One example of a suitable hyperspectral sensor is the AISA hyperspectral sensor, which is an advanced imaging spectrometer. The AISA sensor measures electromagnetic energy over the visible and NIR spectral bands, from 430 nm to 910 nm. The AISA sensor includes a “push broom” type of sensor, meaning that it can scan a single line at a time, and has a spectral resolution of 2.9 nm and a 20-degree field of vision. An AISA hyperspectral sensor does not include an integrated polarizer, but such a polarizer can optionally be included external to the AISA hyperspectral sensor.

Other types of sensors can also be used, that collect light from the region(s) 60 in other orders. For example, light can be obtained and/or spectrally resolved concurrently from all region(s) 60. Or, for example, the light from each region(s) 60 can be obtained separately. Or, for example, the light from a subset of the region(s) 60 can be obtained concurrently, but at a different time from light from other subsets of the region(s) 60. Or, for example, a portion of the light from all the region(s) 60 can be obtained concurrently, but at a different time from other portions of the light from all the region(s) 60 (for example, the intensity of a particular wavelength from all region(s) 60 can be measured concurrently, and then the intensity of a different wavelength from all region(s) 60 can be measured concurrently).

Additionally or alternatively, some embodiments can include a liquid crystal tunable filter (LCTF) based hyperspectral sensor. An LCTF-based sensor obtains light from all region(s) 60 at a time, within a single narrow spectral band at a time. The LCTF-based sensor selects the single band by applying an appropriate voltage to the liquid crystal tunable filter, and recording a map of the reflected intensity of the region(s) 60 at that band. The LCTF-based sensor then sequentially selects different spectral bands by appropriately adjusting the applied voltage, and recording corresponding maps of the reflected intensity of the region(s) 60 at those bands. Another suitable type of sensor is a “whisk-broom” sensor that concurrently collects spectra from both columns and rows of region(s) 60 in a pre-defined pattern. Not all systems use a scan mirror to obtain light from the subject. For example, an LCTF-based sensor concurrently obtains light from all region(s) 60 at a time, so scanning the subject is not necessary.

It is appreciated that other types of hyperspectral sensing devices may be used as the field sensor(s) 56 without departing from the scope of the present disclosure. Moreover, the field sensor(s) 56 may additionally or alternatively correspond to a LIDAR system, which may be used for three-dimensional imaging, a digital camera, a terahertz sensor, and/or any other practicable sensor.

In addition, one or more environmental sensors 76 may be operably coupled and/or communicatively coupled with the vehicle 10 and configured to generate data indicative of various environmental conditions. The environmental sensors 76 can include, for example, one or more ambient temperature sensors, an ambient pressure sensor, a humidity sensor, and/or any other practicable sensor.

In some cases, a computing system 110 (FIG. 3) may be configured to identify one or more zones 74 of the field 20 having a moisture content that exceeds a defined moisture content. When the vehicle 10 approaches the zone 74, the computing system 110 may calculate a probability of the vehicle 10 experiencing tractive element slippage, which may negatively affect the field 20 and/or lead to the vehicle 10 getting stuck or generally failing to continue traversing the field 20 at a defined speed based on the moisture content, one or more environmental conditions, one or more machine conditions, one or more machine configurations, including whether the vehicle 10 is operably coupled with any additional ground-contacting components, such as one or more ground-engaging tools, and/or any other factor. If the probability of the vehicle 10 experiencing tractive element slippage exceeds a defined probability value, the system 100 may be configured to generate a control command to navigate the vehicle 10 around the zone 74 to mitigate the possibility of the vehicle 10 experiencing tractive element slippage.

It will be appreciated that the configuration of the agricultural vehicle 10 described above and shown in FIGS. 1 and 2 are provided only to place the present subject matter in an example field of use. Thus, it will be appreciated that the present subject matter may be readily adaptable to any manner of vehicle configuration, including any suitable vehicle configuration and/or implement configuration.

Referring now to FIG. 3, a system 100 for an agricultural operation, according to various examples, may generally include a first vehicle 10-1 and a second vehicle 10-2. In some instances, the first vehicle 10-1 may be capable of capturing data associated with the field 20. In turn, the second vehicle 10-2 may be configured to apply an agricultural product to the field 20 (or perform any other agricultural operation). In some cases, the operation of one or more components of the second vehicle 10-2, which may affect the trajectory of the second vehicle 10-2, the speed of the second vehicle 10-2, etc., may be determined or altered based at least in part on the data captured by the first vehicle 10-1. Additionally or alternatively, the operation of one or more components of the second vehicle 10-2 may be determined or altered based at least in part on additional data that is captured during the operation of the second vehicle 10-2. Alternatively, the operation of one or more components of the second vehicle 10-2 may be determined or altered based at least in part on data that is captured solely during the operation of the second vehicle 10-2. For example, one field sensor(s) 56 may be used to collect data associated with a reflectivity value of the soil within the field 20. The measured reflectivity values may then be used as input into a model (e.g., a machine-learned model) for identifying one or more zones 74 of the field 20 having a moisture content that exceeds a defined moisture content within the field 20. Based on the moisture content within the identified one or more zones 74 of the field 20 having a moisture content that exceeds a defined moisture content, one or more control actions may be generated by a computing system 110. Additionally, in some cases, the environmental sensor(s) 76 may provide data indicative of an ambient temperature, which may also be used as an input into the model, as an ambient temperature may affect whether the vehicle 10 may be capable of traversing the one or more zones 74. For instance, while a moisture contact within a zone 74 may exceed a defined moisture content, if an ambient temperature is below a lower temperature threshold (e.g., twenty degrees Fahrenheit), the vehicle 10 may be capable of traversing the zone 74.

In the illustrated example, the first vehicle 10-1 is configured as one or more unmanned aerial vehicles (UAVs) 10-1 configured to be flown over the field 20 to allow data to be collected via a field sensor(s) 56 and/or an environmental sensor(s) 76 supported on the UAV 10-1. While the first vehicle 10-1 is illustrated and described as a UAV, it will be appreciated that the first vehicle 10-1 may additionally or alternatively be configured as a tractor, a harvester, a self-propelled windrower, a self-propelled sprayer, and/or the like. In addition, it will be appreciated that the first vehicle 10-1 may be human-controlled, autonomously controlled, and/or semi-autonomously controlled without departing the scope of the present disclosure.

In several embodiments, the UAV 10-1 may be flown across the field 20 to allow the one or more field sensor(s) 56 and/or an environmental sensor(s) 76 to collect data associated with a reflectivity value of the soil within the field 20.

In addition to the one or more field sensor(s) 56 and/or an environmental sensor(s) 76, the UAV 10-1 may also support one or more additional components, such as an onboard controller 108. In general, the UAV controller 108 may be configured to control the operation of the UAV 10-1, such as by controlling the propulsion system 134 of the UAV 10-1 to cause the UAV 10-1 to be moved relative to the field 20. For instance, in some embodiments, the UAV controller 108 may be configured to receive flight plan data associated with a proposed flight plan for the UAV 10-1, such as a flight plan selected such that the UAV 10-1 makes one or more passes across the field 20 in a manner that allows the one or more field sensor(s) 56 and/or an environmental sensor(s) 76 to capture data across at least a portion of the field 20. It should be appreciated that the UAV 10-1 may generally correspond to any suitable aerial vehicle capable of unmanned flight, such as any UAV capable of controlled vertical, or nearly vertical, takeoffs and landings. For instance, in the illustrated embodiment, the UAV 10-1 corresponds to a quadcopter. However, in other embodiments, the UAV 10-1 may correspond to any other multi-rotor aerial vehicle, such as a tricopter, hexacopter, or octocopter. In still further embodiments, the UAV 10-1 may be a single-rotor helicopter, or a fixed-wing, hybrid vertical takeoff, and landing aircraft. Still further, it will be appreciated that the first vehicle 10-1 may be implemented as any other vehicle capable of performing any of the functions described herein without departing from the scope of the present disclosure.

Moreover, in some embodiments, the second vehicle 10-2 may correspond to the agricultural vehicle 10 described herein. Alternatively, the vehicle 10 may correspond to any other suitable vehicle configured to apply or deliver an agricultural product to the field 20, such as a granular fertilizer applicator, etc. In some cases, a computing system 110 may be configured to identify one or more zones 74 of the field 20 having a moisture content that exceeds a defined moisture content.

When the second vehicle 10-2 approaches the zone 74, the computing system 110 may calculate a probability of the vehicle experiencing tractive element slippage based on the moisture content, one or more machine conditions, one or more environmental conditions, one or more machine configurations, and/or any other factor. In some cases, the one or more machine configurations can include the presence of one or more implements operably coupled with the vehicle 10, which in combination, may be referred to herein as the vehicle 10. In such cases, the computing system 110 may calculate the probability of the vehicle experiencing tractive element slippage based at least in part on the presence and/or type of one or more ground-contacting components, such as a ground engaging tool (e.g., tillage tools), being operably coupled with the vehicle 10. For example, the computing system may use a first probability factor when the vehicle is operably coupled with a tillage implement that includes one more disks and a second probability factor when the tillage implement includes one or more shanks. In various examples, the computing system 110 may implement machine learning engine methods and algorithms that utilize one or several machine learning techniques including, for example, decision tree learning, including, for example, random forest or conditional inference trees methods, neural networks, support vector machines, clustering, and Bayesian networks. These algorithms can include computer-executable code that may be used to generate a predictive evaluation of the probability factors. In some instances, the machine learning engine may allow for changes to the probability factors based on the machine configurations and conditions to be updated without human intervention.

If the probability of the vehicle experiencing tractive element slippage exceeds a defined probability value, the system 100 may be configured to generate a control command to navigate the second vehicle 10-2 around the zone 74 to mitigate the possibility of the second vehicle 10-2 experiencing tractive element slippage, which may negatively affect the field 20 and/or lead to the second vehicle 10-2 getting stuck or generally failing to continue traversing the field 20 at a defined speed.

Additionally, as shown in FIG. 3, the disclosed system 100 may also include one or more remote computing systems 110 separate from or remote to the UAV 10-1. In several embodiments, the one or more remote computing systems 110 may be communicatively coupled to the UAV controller 108 to allow data to be transmitted between the UAV 10-1 and the one or more remote computing systems 110. For instance, in various embodiments, the one or more remote computing systems 110 may be configured to transmit instructions or data to the UAV controller 108 associated with the desired flight plan across the field 20. Similarly, the UAV controller 108 may be configured to transmit or deliver the data collected by the one or more field sensor(s) 56 and/or an environmental sensor(s) 76 to the one or more remote computing systems 110.

The one or more remote computing systems 110 may correspond to a stand-alone component or may be incorporated into or form part of a separate component or assembly of components. For example, in various embodiments, the one or more remote computing systems 110 may form part of a base station 112. In such an embodiment, the base station 112 may be disposed at a fixed location, such as a farm building or central control center, which may be proximal or remote to the field 20, or the base station 112 may be portable, such as by being transportable to a location within or near the field 20. In addition to the base station 112 (or an alternative thereto), the one or more remote computing systems 110 may form part of an agricultural vehicle, such as the agricultural vehicle 10 described above (e.g., a sprayer, granular fertilizer applicator, etc.). For instance, the one or more remote computing systems 110 may correspond to a vehicle controller provided in operative association with the second vehicle 10-2 and/or an implement controller provided in operative association with a corresponding implement of the second vehicle 10-2.

In other embodiments, the one or more remote computing systems 110 may correspond to or form part of a remote cloud-based system 114. For instance, the first vehicle 10-1, the second vehicle 10-2, the base station 112, and/or an electronic device 116 may be communicatively coupled with one another and/or one or more remote sites, such as a remote server 118 via a network/cloud 120 to provide data and/or other information therebetween. The network/cloud 120 represents one or more systems by which the first vehicle 10-1, the second vehicle 10-2, the base station 112, and/or the electronic device 116 may communicate with the remote server 118. The network/cloud 120 may be one or more of various wired or wireless communication mechanisms, including any desired combination of wired and/or wireless communication mechanisms and any desired network topology (or topologies when multiple communication mechanisms are utilized). Example communication networks 120 include wireless communication networks (e.g., using Bluetooth, IEEE 802.11, etc.), local area networks (LAN), and/or wide area networks (WAN), including the Internet and the Web, which may provide data communication services and/or cloud computing services. The Internet is generally a global data communications system. It is a hardware and software infrastructure that provides connectivity between computers. In contrast, the Web is generally one of the services communicated via the Internet. The Web is generally a collection of interconnected documents and other resources, linked by hyperlinks and URLs. In many technical illustrations when the precise location or interrelation of Internet resources are generally illustrated, extended networks such as the Internet are often depicted as a cloud (e.g. 302 in FIG. 3). The verbal image has been formalized in the newer concept of cloud computing. The National Institute of Standards and Technology (NIST) defines cloud computing as “a model for enabling convenient, on-demand network access to a shared pool of configurable computing resources (e.g., networks, servers, storage, applications, and services) that can be rapidly provisioned and released with minimal management effort or service provider interaction.” Although the Internet, the Web, and cloud computing are not the same, these terms are generally used interchangeably herein, and they may be referred to collectively as the network/cloud 120.

The server 118 may be one or more computing devices, each of which may include at least one processor and at least one memory, the memory storing instructions executable by the processor, including instructions for carrying out various steps and processes. The server 118 may include or be communicatively coupled to a data store 122 for storing collected data as well as instructions for the first vehicle 10-1, the second vehicle 10-2, the base station 112, and/or the electronic device 116 with or without intervention from a user, the first vehicle 10-1, the second vehicle 10-2, the base station 112, and/or the electronic device 116. Moreover, the server 118 may be capable of analyzing initial or raw sensor data received from the first vehicle 10-1, the second vehicle 10-2, the electronic device 116, and/or the base station 112, and final or post-processing data (as well as any intermediate data created during data processing). Accordingly, the instructions provided to any one or more of the first vehicle 10-1, the second vehicle 10-2, the base station 112, and/or the electronic device 116 may be determined and generated by the server 118 and/or one or more cloud-based applications 124. In such instances, a user interface of the first vehicle 10-1, a user interface 32 of the second vehicle 10-2, and/or the electronic device 116 may be a dummy device that provides various notifications based on instructions from the network/cloud 120.

With further reference to FIG. 3, the server 118 also generally implements features that may enable the first vehicle 10-1, the second vehicle 10-2, the base station 112, and/or the electronic device 116 to communicate with cloud-based applications 124. Communications from the electronic device 116 can be directed through the network/cloud 120 to the server 118 and/or cloud-based applications 124 with or without a networking device, such as a router and/or modem. Additionally, communications from the cloud-based applications 124, even though these communications may indicate one of the first vehicle 10-1, the second vehicle 10-2, the base station 112, and/or the electronic device 116 as an intended recipient, can also be directed to the server 118. The cloud-based applications 124 are generally any appropriate services or applications 124 that are accessible through any part of the network/cloud 120 and may be capable of interacting with the electronic device 116.

In various examples, the first vehicle 10-1, the second vehicle 10-2, the base station 112, and/or the electronic device 116 can be feature-rich with respect to communication capabilities, i.e. have built-in capabilities to access the network/cloud 120 and any of the cloud-based applications 124 or can be loaded with, or programmed to have such capabilities. The first vehicle 10-1, the second vehicle 10-2, the base station 112, and/or the electronic device 116 can also access any part of the network/cloud 120 through industry-standard wired or wireless access points, cell phone cells, or network nodes. In some examples, users can register to use the remote server 118 through the first vehicle 10-1, the second vehicle 10-2, the base station 112, and/or the electronic device 116, which may provide access to the first vehicle 10-1, the second vehicle 10-2, the base station 112, and/or the electronic device 116 and/or thereby allow the server 118 to communicate directly or indirectly with the first vehicle 10-1, the second vehicle 10-2, the base station 112, and/or the electronic device 116. In various instances, the first vehicle 10-1, the second vehicle 10-2, the base station 112, and/or the electronic device 116 may also communicate directly, or indirectly, with the first vehicle 10-1, the second vehicle 10-2, the base station 112, and/or the electronic device 116 or one of the cloud-based applications 124 in addition to communicating with or through the server 118. According to some examples, the first vehicle 10-1, the second vehicle 10-2, the base station 112, and/or the electronic device 116 can be preconfigured at the time of manufacture with a communication address (e.g. a URL, an IP address, etc.) for communicating with the server 118 and may or may not have the ability to upgrade or change or add to the preconfigured communication address.

Referring still to FIG. 3, when a new cloud-based application 124 is developed and introduced, the server 118 can be upgraded to be able to receive communications for the new cloud-based application 124 and to translate communications between the new protocol and the protocol used by the first vehicle 10-1, the second vehicle 10-2, the base station 112, and/or the electronic device 116. The flexibility, scalability, and upgradeability of current server technology render the task of adding new cloud-based application protocols to the server 118 relatively quick and easy.

In several embodiments, an application interface 126 may be operably coupled with the cloud 302 and/or the application 124. The application interface 126 may be configured to receive data related to the first vehicle 10-1, the second vehicle 10-2, the base station 112, and/or the electronic device 116. In various embodiments, one or more inputs related to the field data may be provided to the application interface 126. For example, a farmer, a vehicle user, a company, or other persons may access the application interface 126 to enter the inputs related to the field data. Additionally or alternatively, the inputs related to the field data may be received from a remote server 118. For example, the inputs related to the field data may be received in the form of software that can include one or more objects, agents, lines of code, threads, subroutines, databases, application programming interfaces (APIs), or other suitable data structures, source code (human-readable), object code (machine-readable). In response, the system 100 may update any input/output based on the received inputs. The application interface 126 can be implemented in hardware, software, or a suitable combination of hardware and software, and it can be one or more software systems operating on a general-purpose processor platform, a digital signal processor platform, or other suitable processors.

In some examples, at various predefined periods and/or times, the first vehicle 10-1, the second vehicle 10-2, the base station 112, and/or the electronic device 116 may communicate with the server 118 through the network/cloud 120 to obtain the stored instructions, if any exist. Upon receiving the stored instructions, the first vehicle 10-1, the second vehicle 10-2, the base station 112, and/or the electronic device 116 may implement the instructions. In some instances, the first vehicle 10-1, the second vehicle 10-2, the base station 112, and/or the electronic device 116 can send event-related data to the server 118 for storage in the data store 122. This collection of event-related data can be accessed by any number of users, the first vehicle 10-1, the second vehicle 10-2, the base station 112, and/or the electronic device 116 to assist with application processes.

In some instances, the electronic device 116 may also access the server 118 to obtain information related to stored events. The electronic device 116 may be a mobile device, tablet computer, laptop computer, desktop computer, watch, virtual reality device, television, monitor, or any other computing device or another visual device.

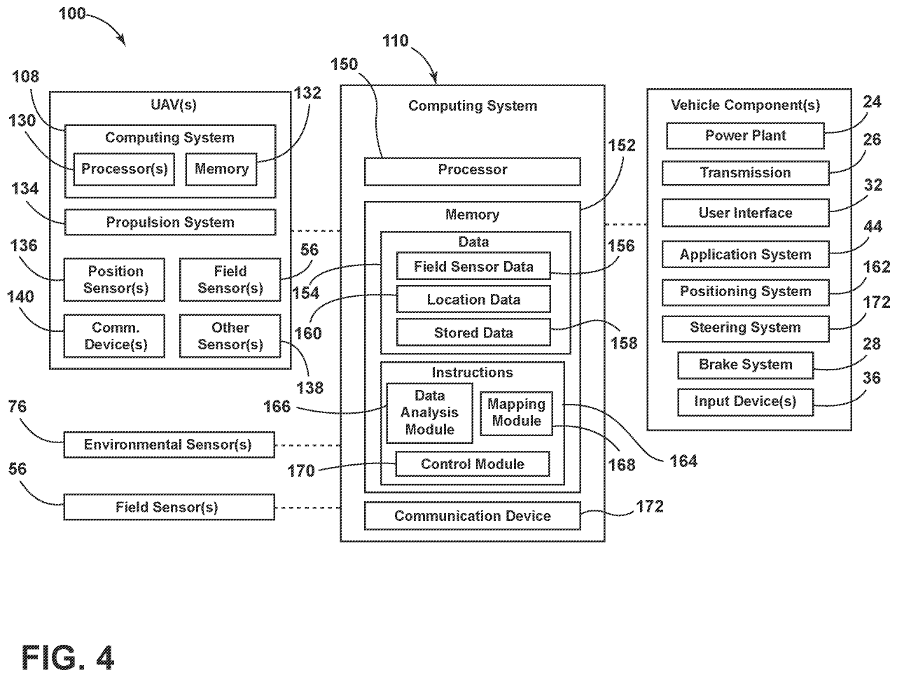

In various embodiments, the data used by the first vehicle 10-1, the second vehicle 10-2, the electronic device 116, the remote server 118, the data store 122, the application 124, the application interface 126, the electronic device 116, and/or any other component described herein for any purpose may be based on data provided by the one or more sensors operably coupled with the first vehicle 10-1 (e.g., the field sensor(s) 56, an environmental sensor 76 (FIG. 4) or other sensor (FIG. 4)), the one or more sensors operably coupled with the second vehicle 10-2 (e.g., the field sensor 56, an environmental sensor 76 (FIG. 4), etc.), and/or third-party data that may be converted into comparable data that may be used independently or in conjunction with data collected from the one or more sensors operably coupled with the first vehicle 10-1 and/or the one or more sensors operably coupled with the second vehicle 10-2.

In various embodiments, based on the data collected during a first operation performed by the first vehicle 10-1, the system 100 may be configured to identify one or more zones 74 of the field 20 having a moisture content that exceeds a defined moisture content. When the second vehicle 10-2 approaches the zone 74, the system 100 may calculate a probability of the vehicle experiencing tractive element slippage based on the moisture content, one or more environmental conditions, one or more machine conditions, one or more machine configurations, and/or any other factor. If the probability of the vehicle experiencing tractive element slippage exceeds a defined probability value, the system 100 may be configured to generate a control command to navigate the second vehicle 10-2 around the zone 74 to mitigate the possibility of the second vehicle 10-2 experiencing tractive element slippage, which may negatively affect the field 20 and/or lead to the vehicle getting stuck or generally failing to continue traversing the field 20 at a defined speed.

In various examples, the server 118 may implement machine learning engine methods and algorithms that utilize one or several machine learning techniques including, for example, decision tree learning, including, for example, random forest or conditional inference trees methods, neural networks, support vector machines, clustering, and Bayesian networks. These algorithms can include computer-executable code that can be retrieved by the server 118 through the network/cloud 120 and may be used to generate a predictive evaluation of the field 20. In some instances, the machine learning engine may allow for changes to a map of the field 20 to be updated without human intervention.

Referring now to FIG. 4, a schematic view of the UAV 10-1 and the remote computing system 110 are illustrated in accordance with aspects of the present disclosure. It should be appreciated, however, that, in other embodiments, the disclosed system 100 may have any other suitable system configuration or architecture and/or may incorporate any other suitable components and/or combination of components that generally allow the system 100 to function as described herein.

As shown, the system 100 may include one or more UAVs, such as the UAV 10-1 described above with reference to FIG. 3. In general, the UAV 10-1 may include and/or be configured to support various components, such as one or more sensors, controllers, and propulsion systems. For instance, as indicated above, the UAV 10-1 may be provided in operative association with one or more field sensor(s) 56 configured to capture or collect data associated with the field 20 over which the UAV 10-1 is being flown and/or an environmental sensor(s) 76 configured to capture or collect data associated with environmental conditions surrounding the field 20.

Additionally, as indicated above, the UAV 10-1 may also include a controller 108. In general, the UAV controller 108 may correspond to any suitable processor-based device(s), such as a computing device or any combination of computing devices. Thus, in several embodiments, the UAV controller 108 may include one or more processor(s) 130 and associated memory 132 configured to perform a variety of computer-implemented functions. As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory 132 of the UAV controller 108 may generally comprise memory element(s) including, but not limited to, computer-readable medium (e.g., random access memory (RAM)), computer-readable non-volatile medium (e.g., a flash memory), a compact disc-read only memory (CD-ROM), a magneto-optical disk (MOD), a digital versatile disc (DVD) and/or other suitable memory elements. Such memory 132 may generally be configured to store suitable computer-readable instructions that, when implemented by the processor(s) 130, configure the UAV controller 108 to perform various computer-implemented functions. It should be appreciated that the UAV controller 108 may also include various other suitable components, such as a communications circuit or module, a network interface, one or more input/output channels, a data/control bus, and/or the like.

In several embodiments, the UAV controller 108 may be configured to control the operation of a propulsion system 134 of the UAV 10-1. For instance, as indicated above, the UAV controller 108 may be configured to control the propulsion system 134 in a manner that allows the UAV 10-1 to be flown across a field 20 according to a predetermined or desired flight plan. In this regard, the propulsion system 134 may include any suitable components that allow for the operation of one or more components, speed, and/or altitude of the UAV 10-1 to be regulated, such as one or more power sources (e.g., one or more batteries), one or more drive sources (e.g., one or more motors and/or engines), and one or more lift/steering sources (e.g., propellers, blades, wings, rotors, and/or the like).

Additionally, as shown in FIG. 4, the UAV 10-1 may also include one or more positioning device(s) 136. In various embodiments, the positioning device(s) 136 may be configured to determine the exact location of the UAV 10-1 within the field 20 using a satellite navigation position system (e.g. a GPS, a Galileo positioning system, the Global Navigation satellite system (GLONASS), the BeiDou Satellite Navigation and Positioning system, and/or the like) and/or a dead reckoning device. In such embodiments, the location determined by the positioning device(s) 136 may be transmitted to the UAV controller 108 (e.g., in the form coordinates) and stored within the controller's memory for subsequent processing and/or analysis. By monitoring the location of the UAV 10-1 as a pass is being made across the field 20, the sensor data acquired via the one or more field sensor(s) 56 and/or an environmental sensor(s) 76 may be geo-located within the field 20. For instance, in various embodiments, the location coordinates derived from the positioning device(s) 136 and the sensor data generated by the one or more field sensor(s) 56 and/or an environmental sensor(s) 76 may both be time-stamped. In such an embodiment, the time-stamped data may allow the sensor data to be matched or correlated to a corresponding set of location coordinates received or derived from the positioning device(s) 136, thereby allowing a field map to be generated that locates various objects, such as and landmarks, within the field 20 relative to one another.

It should be appreciated that the UAV 10-1 may also include any other suitable components. For instance, in addition to the one or more field sensor(s) 56 and/or an environmental sensor(s) 76, the UAV 10-1 may also include various other sensors 138, such as one or more inertial measurement units for monitoring the orientation of the UAV 10-1 and/or one or more altitude sensors for monitoring the pose of the UAV 10-1 relative to the ground. As used herein, “pose” includes the position and orientation of an object, such as the position and orientation of a vehicle, in some reference frame. Moreover, the UAV 10-1 may include a communications device(s) 140 to allow the UAV controller 108 to be communicatively coupled to one or more other system components. The communications device 140 may, for example, be configured as a wireless communications device (e.g., an antenna or transceiver) to allow for the transmission of wireless communications between the UAV controller 108 and one or more other remote system components.

As shown in FIG. 4, the system 100 may also include one or more computing systems 110 or controllers remote to the UAV 10-1, such as the one or more remote computing systems 110 described above with reference to FIG. 3. In general, the one or more remote computing systems 110 may be configured to be in communication with one or more components of the UAV 10-1 to allow data to be transferred between the UAV 10-1 and the one or more remote computing systems 110, such as sensor data collected via the one or more field sensor(s) 56 and/or an environmental sensor(s) 76. As indicated above, the one or more remote computing systems 110 may correspond to a stand-alone component or may be incorporated into or form part of a separate component or assembly of components. For example, the one or more remote computing systems 110 may be incorporated into or form part of a base station 112 and/or a cloud computing system 110. In addition, or as an alternative thereto, the one or more remote computing systems 110 may correspond to a component of the second vehicle 10-2 and/or an associated implement towed by the second vehicle 10-2, such as by corresponding to a vehicle controller and/or an implement controller.

Similar to the UAV controller 108, the one or more remote computing systems 110 may be configured as any suitable processor-based device(s), such as a computing device or any combination of computing devices. As such, the one or more remote computing systems 110 may include one or more processor(s) 150 and associated memory 152 configured to perform a variety of computer-implemented functions. The memory 152 may generally be configured to store suitable computer-readable instructions that, when implemented by the processor(s) 150, configure the one or more remote computing systems 110 to perform various computer-implemented functions. It should be appreciated that the one or more remote computing systems 110 may also include various other suitable components, such as a communications circuit or module, a network interface, one or more input/output channels, a data/control bus, and/or the like.

In various embodiments, the memory 152 of the one or more remote computing systems 110 may include one or more databases 154 for storing information received and/or generated by the computing system 110. For instance, as shown in FIG. 4, the memory 152 may include a field sensor(s) database 156 storing data associated with the field data captured by the field sensor(s) 56, including the captured data and/or data deriving from the captured data (e.g., disparity maps, depth images generated based on the captured data by the field sensor(s) 56, etc.) and/or an environmental sensor(s) 76. Additionally, the memory 152 may include a stored data database 158 storing data acquired from various sources. For instance, the stored data can include a map that is generated through any method, such as with a previous agricultural operation, user-entered information, from the one or more field sensor(s) 56 and/or an environmental sensor(s) 76, and/or other systems that previously identified (and possibly, verified) zones 74. Additionally or alternative, the stored data may include a slippage factor of the second vehicle 10-2 that is equal to a summation of each of the current vehicle conditions and configuration.

Additionally or alternatively, as shown in FIG. 4, the memory 152 may also include a location database 160, which may be configured to store location data generated by a positioning system 162 that is stored in association with the field data for later use in geo-locating the field data relative to the field 20. In some embodiments, the positioning system 162 may be configured as a satellite navigation positioning device (e.g. a GPS, a Galileo positioning system, a Global Navigation satellite system (GLONASS), a BeiDou Satellite Navigation and Positioning system, a dead reckoning device, and/or the like) to determine the location of the vehicle 10.

Moreover, as shown in FIG. 4, in several embodiments, the memory 152 may also include instructions 164 that may be executed by the processor 150 to implement a data analysis module 166. In general, the data analysis module 166 may be configured to process/analyze the captured data received from the field sensor(s) 56 and/or an environmental sensor(s) 76, the stored data, and/or the location data. In several embodiments, the data analysis module 166 may be configured to execute one or more data processing algorithms to identify one or more zones 74 of the field having a moisture content that exceeds a defined moisture content. When the vehicle 10-2 approaches the zone 74, the computing system 110 may calculate a probability of the vehicle 10-2 experiencing tractive element slippage based on the moisture content, one or more environmental conditions, one or more machine conditions, one or more machine configurations, and/or any other factor, which may be based at least in part on a current condition and configuration of the power plant 24, the transmission system 26, the application system, a steering system and/or any other system. If the probability of the vehicle 10-2 experiencing tractive element slippage exceeds a defined probability value, the system 100 may be configured to generate a control command to navigate the second vehicle 10-2 around the zone 74 to mitigate the possibility of the second vehicle 10-2 experiencing tractive element slippage, which may negatively affect the field 20 and/or lead to the vehicle 10-2 getting stuck or generally failing to continue traversing the field 20 at a defined speed.

Moreover, the instructions stored within the memory 152 of the computing system 110 may be executed by the processor(s) 150 to implement a mapping module 168 that is configured to generate one or more maps of the field 20 based on the field data. It should be appreciated that, as used herein, a “map” may generally correspond to any suitable dataset that correlates data to various locations within a field 20. Thus, for example, a map may simply correspond to a data table that correlates field contour or topology data to various locations within the field 20 or may correspond to a more complex data structure, such as a geospatial numerical model that can be used to determine a position of each identified zone 74 within the field 20, which may, for instance, then be used to generate a graphically displayed map or visual indicator.

Referring still to FIG. 4, in some embodiments, the instructions 216 stored within the memory 212 of the computing system 110 may also be executed by the processor(s) 150 to implement a control module 170. In general, the control module 170 may be configured to electronically control the operation of one or more components of the second vehicle 10-2. For instance, when a control command is generated based on the probability of the vehicle 10-2 experiencing tractive element slippage exceeding a defined probability value, the control module 170 may alter at least one of the power plant 24, the transmission system 26, and/or a steering system of the vehicle 10-2 to avoid the identified zone 74. Additionally or alternatively, the control module 170 may generate information that is to be displayed on one or more user interfaces 32 regarding the identified zones 74 of the field 20.

In some instances, the data analysis module 166 may be configured to input the measured reflectivity values received from the field sensors 56 and data indicative of an ambient temperature received from the environmental sensor(s) 76 into a model (e.g., a machine-learned model) for identifying one or more zones 74 of the field 20 to determine one or more avoidance zones within the field 20 (FIG. 1). In such instances, the model may identify one or more zones 74 of the field 20 having a moisture content that exceeds a defined moisture content. In addition, the model may use the ambient temperature data to determine whether the one or more zones 74 of the field 20 having a moisture content that exceeds a defined moisture content may still be traversed. For instance, when an ambient temperature is below a lower temperature threshold (e.g., twenty degrees Fahrenheit), the vehicle 10 may be capable of traversing the zone 74 and the zone 74 may be frozen. As such, the probability of the vehicle 10-2 experiencing tractive element slippage may be lower than a defined probability value. As such, the vehicle 10 may traverse the zone 74. However, when the ambient temperature exceeds the lower temperature threshold (e.g., twenty degrees Fahrenheit), a control action may be generated as the probability of the vehicle 10 experiencing tractive element slippage exceeding a defined probability value within the one or more zones 74.

Additionally or alternatively, the computing system 100 may determine a moisture threshold based at least in part on the presence and/or type of one or more ground-contacting components, such as a ground engaging tool (e.g., tillage tools), being operably coupled with the vehicle 10. For example, each type of ground-engaging tool may have a respective threshold associated with that tool that defines an appropriate moisture range for operating the respective ground-engaging tool. For example, a disk may plug up easier with wet soil than other ground-engaging tools. Likewise, a field cultivator may turn up mud balls when running in wet soil. In some cases, multiple ground-engaging tools may be implemented. In such instances, the computing system 110 may determine a combined moisture threshold. Based on the captured data received from the field sensor(s) 56 and/or an environmental sensor(s) 76, the stored data, the location data, and/or the moisture threshold, the computing system 110 may determine the one or more identified zones 74. In turn, the computing system 110 may generate a control action associated with the one or more identified zones 74.

In various examples, the system 100 may combine the one or more maps of the field 20, which may include the one or more identified zones 74, with data from the one or more environmental sensors 76. In such instances, when the ambient temperature exceeds an upper temperature threshold (e.g., eighty degrees Fahrenheit), the vehicle may return to the avoided zones to perform an agricultural operation in the zones 74 as there may be a higher probability that the moisture content has been reduced to a level below the defined moisture content. In various examples, the computing system 110 may implement machine learning engine methods and algorithms that utilize one or several machine learning techniques including, for example, decision tree learning, including, for example, random forest or conditional inference trees methods, neural networks, support vector machines, clustering, and Bayesian networks. These algorithms can include computer-executable code that may be used to generate a predictive evaluation of an amount of time until the moisture content within the one or more zones 74 will be below the defined moisture content. In some instances, the machine learning engine may allow for changes to the amount of time until the moisture content within the one or more zones 74 will be below the defined moisture content to be updated without human intervention.

Further, as shown in FIG. 4, the computing system 110 may also include a communications device(s) 172 to allow for the computing system 110 to communicate with various vehicle components. For instance, one or more communicative links or interfaces (e.g., one or more data buses) may be provided between the communications device(s) 172 and the power plan, the transmission system 26, the user interface 32, the application system, the positioning system, the steering system, and/or any other component.

It should be appreciated that, although the various control functions and/or actions were generally described above as being executed by one of the controllers of the system (e.g., the UAV controller 108 or the one or more remote computing systems 110, such control functions/actions may generally be executed by either of such controllers 108, 110 and/or may be distributed across both of the controllers 108, 110. In another alternative embodiment, the operation of the UAV 10-1 (e.g., the operation of the propulsion system 134) may be controlled by the one or more remote computing systems 110 as opposed to the UAV controller 108.

It will be appreciated that, although the various control functions and/or actions will generally be described herein as being executed by the computing system 110, one or more of such control functions/actions (or portions thereof) may be executed by a separate computing system or may be distributed across two or more computing systems (including, for example, the computing system 110 and a separate computing system). For instance, in some embodiments, the computing system 110 may be configured to acquire data from the field sensor(s) 56 and/or an environmental sensor(s) 76 for subsequent processing and/or analysis by a separate computing system (e.g., a computing system associated with a remote server). In other embodiments, the computing system 110 may be configured to execute the data analysis module 166 to determine and/or monitor one or more surface conditions within the field 20, while a separate computing system (e.g., a vehicle computing system 110 associated with the agricultural vehicle 10) may be configured to execute the control module 170 to control the operation of the agricultural vehicle 10 based on data and/or instructions 164 transmitted from the computing system 110 that are associated with the monitored surface condition(s).

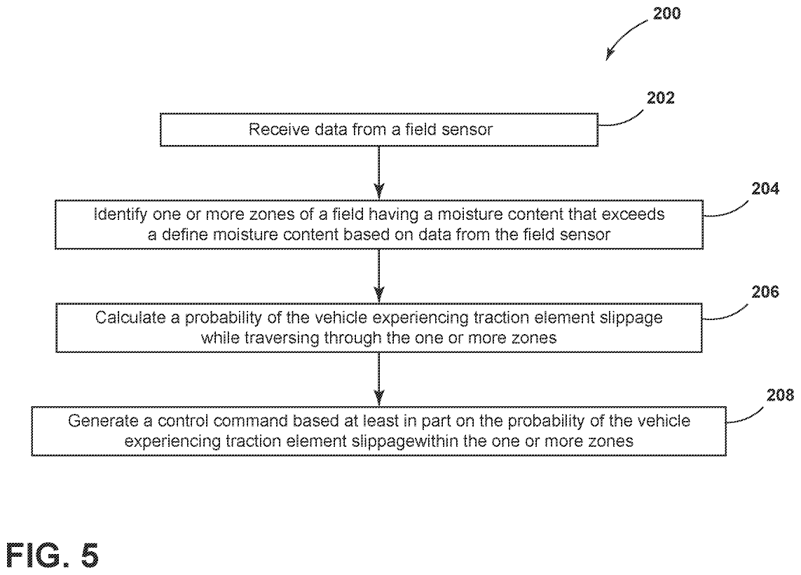

Referring now to FIG. 5, a flow diagram of a method 200 for method for operating an agricultural system is illustrated in accordance with aspects of the present subject matter. In general, the method 200 will be described herein with reference to the agricultural vehicle 10 shown in FIGS. 1 and 2 and the various system components shown in FIGS. 3 and 4. However, it will be appreciated that the disclosed method 200 may be implemented with agricultural vehicles having any other suitable vehicle configurations and/or within systems having any other suitable system configuration. In addition, although FIG. 5 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As illustrated, at (202), the method 200 can include receiving data from a field sensor. In some cases, the field sensor(s) is configured as a hyperspectral sensor. In such instances, the data collected from the hyperspectral sensor may be associated with a reflectivity value of the soil within the field.

At (204), the method 200 can include identifying one or more zones of a field having a moisture content that exceeds a defined moisture content based on data from the field sensor(s) with a computing system. In some cases, the computing system may be configured to identify the one or more zones of the field having a moisture content that exceeds the defined moisture content by inputting the reflectivity values in a machine-learned model.

At (206), the method 200 can include calculating a probability of the vehicle experiencing tractive element slippage while traversing through the one or more zones with the computing system. The method can include receiving a current condition and/or a current configuration of the power plant. In such instances, the probability of the vehicle experiencing tractive element slippage may be based in part on the current condition of the power plant. Additionally or alternatively, the method can include receiving a current condition and/or a current configuration of the transmission system. In such instances, the probability of the vehicle experiencing tractive element slippage may be based in part on the current condition of the power plant. Additionally or alternatively, the method can include receiving a current condition and/or a current configuration of the application system. In such instances, the probability of the vehicle experiencing tractive element slippage may be based in part on the current condition of the application system. Additionally or alternatively, the method can include receiving a current condition and/or a current configuration of the steering system. In such instances, the probability of the vehicle experiencing tractive element slippage may be based in part on the current condition of the steering system.

At (208), the method 200 can include generating a control command based at least in part on the probability of the vehicle experiencing tractive element slippage within the one or more zones with the computing system. In some cases, the control command electronically controls at least one of a power plant, a transmission system, or a steering system of the vehicle to avoid the one or more zones. Additionally or alternatively, the control command can illustrate information related to the one or more zones on a display operably coupled with the computing system.

In various examples, the method 200 may implement machine learning methods and algorithms that utilize one or several machine learning techniques including, for example, decision tree learning, including, for example, random forest or conditional inference trees methods, neural networks, support vector vehicles, clustering, and Bayesian networks. These algorithms can include computer-executable code that can be retrieved by the computing system and/or through a network/cloud and may be used to evaluate and update the boom deflection model. In some instances, the machine learning engine may allow for changes to the boom deflection model to be performed without human intervention.

It is to be understood that the steps of any method disclosed herein may be performed by a computing system upon loading and executing software code or instructions that are tangibly stored on a tangible computer-readable medium, such as on a magnetic medium, e.g., a computer hard drive, an optical medium, e.g., an optical disc, solid-state memory, e.g., flash memory, or other storage media known in the art. Thus, any of the functionality performed by the computing system described herein, such as any of the disclosed methods, may be implemented in software code or instructions that are tangibly stored on a tangible computer-readable medium. The computing system loads the software code or instructions via a direct interface with the computer-readable medium or via a wired and/or wireless network. Upon loading and executing such software code or instructions by the controller, the computing system may perform any of the functionality of the computing system described herein, including any steps of the disclosed methods.

The term “software code” or “code” used herein refers to any instructions or set of instructions that influence the operation of a computer or controller. They may exist in a computer-executable form, such as vehicle code, which is the set of instructions and data directly executed by a computer's central processing unit or by a controller, a human-understandable form, such as source code, which may be compiled in order to be executed by a computer's central processing unit or by a controller, or an intermediate form, such as object code, which is produced by a compiler. As used herein, the term “software code” or “code” also includes any human-understandable computer instructions or set of instructions, e.g., a script, that may be executed on the fly with the aid of an interpreter executed by a computer's central processing unit or by a controller.

This written description uses examples to disclose the technology, including the best mode, and also to enable any person skilled in the art to practice the technology, including making and using any devices or systems and performing any incorporated methods. The patentable scope of the technology is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

What is claimed is:

1. An agricultural system comprising:
a vehicle including one or more ground tractive elements;
a field sensor configured to capture data indicative of a moisture content within a field; and
a computing system communicatively coupled to the field sensor, the computing system including a processor and associated memory, the memory storing instructions that, when implemented by the processor, configure the computing system to:
receive the data from the field sensor;
identify one or more zones of the field having a moisture content that exceeds a defined moisture content;
calculate a probability of the vehicle experiencing tractive element slippage while traversing through the one or more zones; and
generate a control command based at least in part on the probability of the vehicle experiencing tractive element slippage exceeding a defined probability value within the one or more zones.

2. The agricultural system of claim 1, wherein the computing system is further configured to:
receive a current condition of a power plant,
wherein the probability of the vehicle experiencing tractive element slippage is based in part on the current condition of the power plant.

3. The agricultural system of claim 2, wherein the computing system is further configured to:
receive a current condition of a transmission system,
wherein the probability of the vehicle experiencing tractive element slippage is based in part on the current condition of the power plant.

4. The agricultural system of claim 2, wherein the computing system is further configured to:
receive a current condition of an application system,
wherein the probability of the vehicle experiencing tractive element slippage is based in part on the current condition of the application system.

5. The agricultural system of claim 2, wherein the computing system is further configured to:
receive a current condition of a steering system,
wherein the probability of the vehicle experiencing tractive element slippage is based in part on the current condition of the steering system.

6. The agricultural system of claim 1, wherein the control command navigates the vehicle around the one or more zones.

7. The agricultural system of claim 6, wherein the computing system is configured to navigate the vehicle around the one or more zones through electronic control of at least one of a power plant, a transmission system, or a steering system of the vehicle.

8. The agricultural system of claim 6, further comprising:
a display operably coupled with the computing system, the computing system configured to illustrate information related to the one or more zones.

9. The agricultural system of claim 1, wherein the field sensor is configured as a hyperspectral sensor.

10. The agricultural system of claim 9, wherein the data collected from the hyperspectral sensor is associated with a reflectivity value of a soil within the field.

11. The agricultural system of claim 10, wherein the computing system is configured to identify one or more zones of the field having a moisture content that exceeds the defined moisture content by inputting the reflectivity values in a machine-learned model.

12. A method for operating an agricultural system, the method comprising:
receiving data from a field sensor;
identifying, with a computing system, one or more zones of a field having a moisture content that exceeds a defined moisture content based on data from the field sensor; and
calculating, with the computing system, a probability of a vehicle experiencing tractive element slippage while traversing through the one or more zones.

13. The method of claim 12, further comprising:
generating, with the computing system, a control command based at least in part on the probability of the vehicle experiencing tractive element slippage within the one or more zones.

14. The method of claim 13, wherein the control command electronically controls at least one of a power plant, a transmission system, or a steering system of the vehicle to avoid the one or more zones.

15. The method of claim 13, wherein the control command illustrates information related to the one or more zones on a display operably coupled with the computing system.

16. An agricultural system comprising:
a field sensor configured to capture data indicative of a moisture content within a field; and
a computing system communicatively coupled to the field sensor, the computing system including a processor and associated memory, the memory storing instructions that, when implemented by the processor, configure the computing system to:
receive data from the field sensor;
identify one or more zones of the field having a moisture content that exceeds a defined moisture content; and
calculate a probability of a vehicle experiencing tractive element slippage while traversing through the one or more zones.

17. The agricultural system of claim 16, wherein the computing system is further configured to:
generate a control command based at least in part on the probability of the vehicle experiencing tractive element slippage within the one or more zones.

18. The agricultural system of claim 16, wherein the field sensor is configured as a hyperspectral sensor.

19. The agricultural system of claim 18, wherein the data collected from the hyperspectral sensor is associated with a reflectivity value of a soil within the field.

20. The agricultural system of claim 19, wherein the computing system is configured to identify one or more zones of the field having a moisture content that exceeds the defined moisture content by inputting the reflectivity values in a machine-learned model.
